(19) United States
(12) Patent Application Publication
(10) Pub. No.: US 2005/0232389 A1
(43) Pub. Date: Oct. 20, 2005

(54) Operating method for an x-ray system and x-ray system

(75) Inventor: Klaus Klingenbeck-Regn, Nurnberg (DE)

Correspondence Address: SIEMENS CORPORATION, INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/104,891

(22) Filed: Apr. 13, 2005

(30) Foreign Application Priority Data
Apr. 14, 2004 (DE) 10 2004 018 498.4

(51) Int. Cl. G21K 1/12; H05G 1/60; A61B 6/00; G01N 23/00

(52) U.S. Cl. 378/9

(57) ABSTRACT

An x-ray system features a control device (6), a data storage device (12) and two x-ray units. Each x-ray unit features an x-ray source (1,3) and an x-ray detector (2,4) which can be pivoted around a pivot axis (5) and are arranged opposite one another in relation to the pivot axis (5) in each case. An object (10) can be arranged in the area of the pivot axis (5). The control device (6) controls the x-ray units so that they are simultaneously pivoted by the pivot angle (δα,δβ) around the pivot axis (5). In this case images of the object (10) are recorded by means of the x-ray detectors (2,4) at angular positions (αi,βj) and conveyed to the data storage device (12). The pivot angle (δα,δβ) and the angular positions (αi,βj) are determined here such that, on the basis of the recorded images of the object (10), at least one 3D reconstruction of the object (10) can be determined which also occurs afterwards.

### CROSS REFERENCE TO RELATED APPLICATIONS

This application claims priority to the German application No. 10 2004 018 498.4, filed Apr. 14, 2004 which is incorporated by reference herein in its entirety.

### FIELD OF INVENTION

The present invention relates to an operating method for an x-ray system and to an x-ray system.

### BACKGROUND OF INVENTION

A variety of embodiments and operating methods are known for x-ray systems. Corresponding to this, various computer-aided methods for 3D-reconstruction of the object on the basis of images acquired with these x-ray systems and these operating methods are also known.

Thus for example, for an x-ray system with only a single x-ray unit,
it is known that the control device controls the single x-ray unit such that its x-ray source and its x-ray detector are pivoted by a pivot angle around the pivot axis,
that the control device during the pivoting of the x-ray source and of the x-ray detector activates the x-ray unit such that at angular positions of the x-ray detector an image of the object is recorded in each case and conveyed to the data storage device in each case,
with the pivot angle and the angular positions being determined such that a reconstruction of the object can be determined using the images of the object recorded the x-ray detector.

Corresponding to this, a relevant computer-aided transfer procedure for a 3D-reconstruction of the object on the basis of a number of images of the object is also known.

### SUMMARY OF INVENTION

As far as the applicant knows the images are always acquired with a single x-ray unit. Naturally this also applies to the 3D reconstruction determined on the basis of the acquired images.

However, as mentioned above, x-ray systems with two x-ray units are also known. As far as the applicant knows, these are only essentially used to illuminate the object simultaneously from two different directions, so that individual elements of the object can be localized in three-dimensions. An acquisition of images by means of an x-ray system with two x-ray units for at least one later 3D reconstruction is not known to the applicant.

An object of the present invention is thus, using the aforementioned prior art as a starting point, i.e. the x-ray system with two x-ray units, to open up this x-ray system to other uses.

The object is achieved for the operating method in,
that the control device controls the first x-ray unit such that the first x-ray source and the first x-ray detector are pivoted by a first pivot angle around the pivot axis,
the control device, simultaneously to pivoting first x-ray detector and the first x-ray source, controls the second x-ray unit such that the second x-ray source and the second x-ray detector are pivoted by a second pivot angle around the pivot axis,
during the pivoting of the x-ray sources and the x-ray detectors, the control device controls the x-ray units such that for first angular positions, the first x-ray detector captures a first image of the object in each case and conveys it to the data storage device and for second angular positions, the second x-ray detector captures a second image of the object in each case and conveys it to the data storage device
where the pivot angle and the angular positions are established such that, on the basis of the first and second images of the object recorded by the x-ray detectors, at least one 3D reconstruction of the object can be determined.

For the determination method the object is achieved in a corresponding way by a computer determining the at least one 3D reconstruction on the basis of both the first and also the second images, with the first and second images having been recorded in accordance with the procedure described above.

X-ray detectors feature a plurality of detector elements, which as a rule are arranged as a two-dimensional array. Each detector element, often also groups of detector elements, exhibit detector characteristics which often differ from each other and in addition depend on a number of other factors. Recorded images can thus not simply be processed further with organ-specific software. Instead they must be modified beforehand with x-ray detector-specific corrections. In this case this can alternatively be done immediately after the recording of the first and second images, i.e. by the control device. Alternatively this correction can also be left to be undertaken immediately before determination of the 3D reconstruction, i.e. by the computer.

The method of operation in accordance with the invention opens up a multiplicity of procedures not previously possible.

If at least one of the first angular positions matches one of the second angular positions, it is possible for example for the images recorded for the matching angular positions to be compared and a warning message to be output if the images deviate from each other by more than an image deviation limit. Here too this comparison and the possible issuing of the warning message can alternatively be undertaken by the control device or by the computer.

It can be that both the first and also the second pivot angle are less than a limit angle as from which a 3D reconstruction of the object is possible. If in this case the pivot angles do not overlap at least partly and the sum of the pivot angles, where necessary minus an overlapping angle, is at least as large as the limit angle, it is still possible to undertake the 3D reconstruction. The 3D reconstruction determined is in this case a common 3D reconstruction, in which both the first and also the second images are included.

Naturally it is also possible for the first and/or the second pivot angle to be at least as large as the limit angle. In this case it is especially possible, using the first images, to determine a first 3D reconstruction and/or using second images a second 3D reconstruction of the object. A common 3D reconstruction may possibly also be undertaken on the basis of both the first and also the second images.

The angular areas are as a rule not identical—regardless of any similarity of their sizes—. The first and the second pivot angle thus do not overlap at least partly as a rule. In an extreme case it is thus even possible for the first pivot angle and the second pivot angle together to cover a full circle around the pivot axis.

As a rule the x-ray units are not pivoted independently of each other. Instead the x-ray detectors and the x-ray sources assigned to them are pivoted by the control device with a first or a second angular speed around the pivot axis, with the second angular speed being a function of the first angular speed. The second angular speed can in this case in particular be the same as the first angular speed.

The overlapping area of the two angular areas covered by the two x-ray units can be very different. In an extreme case the two angular areas just adjoin each other or overlap only slightly. At the other extreme there is almost complete overlapping.

In the latter case in particular it is possible for a second angular position to be arranged between two directly consecutive first angular positions in each case and conversely for a first angular position to be arranged between two directly consecutive second angular positions. The difference angle of the first angular positions to the immediately adjoining second angular positions and vice versa is in this case prefer ably about the same. With this situation and synchronous pivoting of the two x-ray units at the same angular speed, double the recording density can thus be achieved.

Often examinations of the heart, i.e. of an organ which moves, are undertaken using x-ray systems. With such examinations it is necessary for the first and second angular positions of the control device to be determined on the basis of the trigger sign als derived from the object. The first and second angular positions can be determined in this case by the control alternately with a same phase relationship of with a different phase relationship to the trigger signals.

In the first case, despite unfavorable conditions for image acquisition—the possible time amounts to only around 20% of the heart beat—it is possible to record relatively many first and second images of the heart. This procedure is of advantage especially in connection with an alternating sequence of first and second angular positions and with the creation of a common 3D reconstruction. In the last case it is possible to create a 3D reconstruction for different phase positions of the heart in each case.

To determine more than one 3D reconstruction it can be possible to compare the 3D reconstructions with each other on the computer side and to issue a warning message if the 3D reconstructions deviate from each other by more than one reconstruction deviation limit.

It is possible for the angular speeds of the control device to be modulated on the basis of the trigger signal. This means that it is possible to optimize the number of first and second images that can be recorded or evaluated. In particular it is possible for the angular speeds of the control device to be modulated such that they will be maximized in the area of the mean of the phase relationships to the trigger signal.

### BRIEF DESCRIPTION OF THE DRAWINGS

Further advantages and details can be found in the following description of exemplary embodiments in connection with the drawings. The Figures show the following basic diagrams

FIG. 1 a block diagram of an x-ray system,

FIG. 2 a flowchart,

FIG. 3 to 6 possible pivot movements,

FIG. 7 a block diagram of a computer and

FIGS. 8 and 9 flowcharts.

### DETAILED DESCRIPTION OF INVENTION

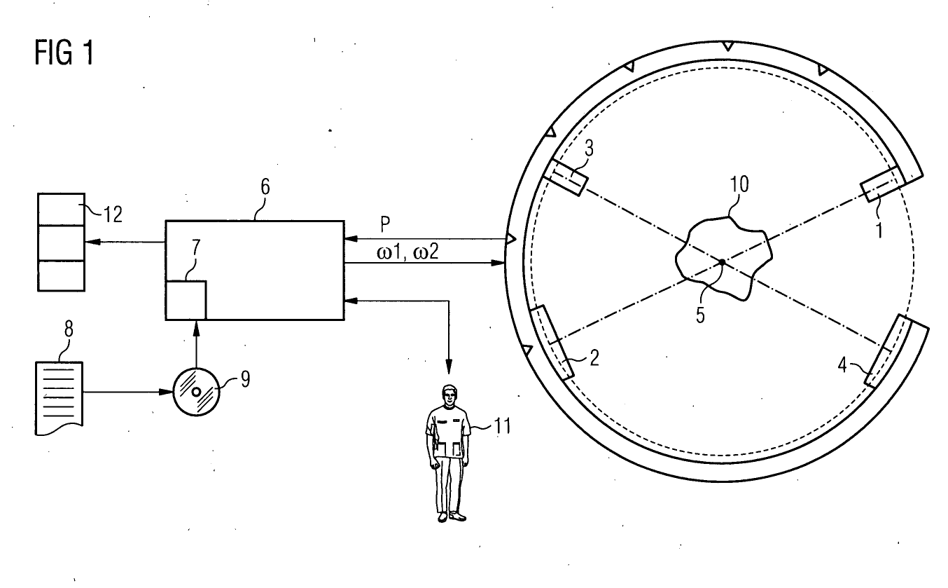

In accordance with FIG. 1 an x-ray system features a first x-ray source 1 and a first x-ray detector 2 as well as a second x-ray source 3 and of a second x-ray detector 4. The first x-ray source 1 and the first x-ray detector 2 form a first x-ray unit the x-ray system. They are arranged opposite one another in relation to a pivot axis 5 and can be pivoted around the pivot axis 5, so that the pivot axis 5 is always arranged between them. Similarly the second x-ray source 3 and the second x-ray detector 4 form a second x-ray unit of the x-ray system. The second x-ray source 3 and the second x-ray detector 4 are also arranged opposite one another in relation to the pivot axis 5 and can be pivoted jointly around the pivot axis 5, so that the pivot axis 5 is always arranged between them.

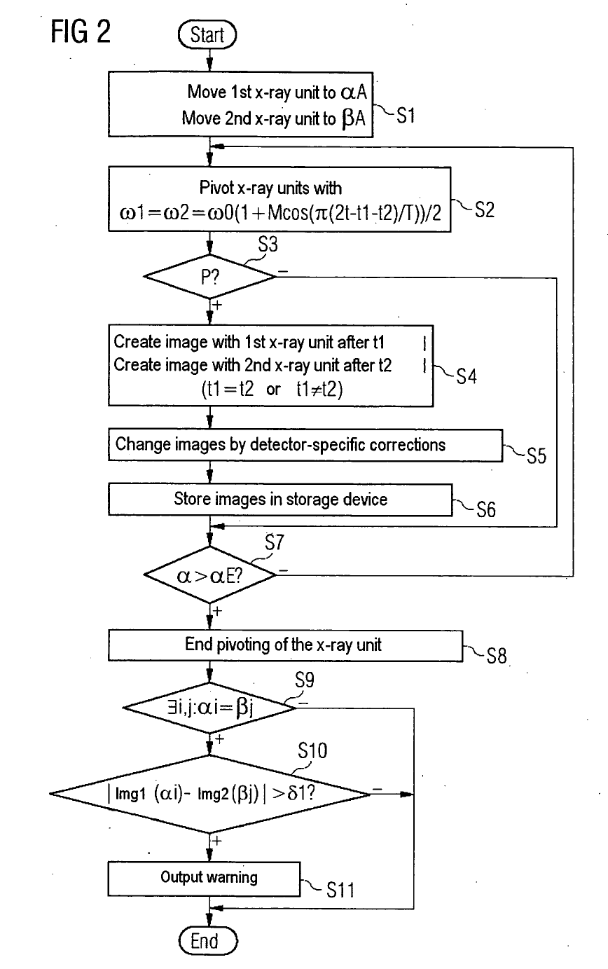

The x-ray system further features a control device 6. The control device 6 is used to control the x-ray unit. It features a program memory 7 in which an operating program 8 is stored. The operating program 8 is conveyed to the control device 6 beforehand in this case via a data medium 9, e.g. a CD-ROM 9, on which the operating program 8 is also stored. In principle the operating program 8 of the control device 6 could however also have been conveyed in another manner, e.g. over a computer network. When the operating program 8 is called, the control device 6 executes an operating method which will be explained in greater detail below in connection with FIG. 2.

To execute the operating method, first of all—see FIG. 1, still outside the actual programmed operating method—an object 10 is arranged in the area of the pivot axis 5. The object 10 can for example be a person whose heart is to be examined. The operating method in accordance with the invention described in greater detail below is not however restricted to examinations of people and also not just to examinations of the heart, but can be used far more universally.

In accordance with FIG. 2, in a step S1, the first x-ray unit is then moved by the control device 6 into its start position, e.g. to a first starting angle αA. Also within the framework of step S1, the second x-ray unit is moved by the control device 6 to a second start angle βA.

Then the control device 6, in a step S2, pivots the first x-ray unit at a first angular speed ω1 and simultaneously the second x-ray unit at a second angular speed ω2 around the pivot axis 5. The second angular speed ω2 in this case is a function of the first angular speed ω1. As a rule the two angular speeds ω1, ω2 are even the same. The units are pivoted up to a first or a second end angle. αE, βE. The x-ray units are thus pivoted by the control device 6 simultaneously by a first or of a second pivot angle δα, δβ around the pivot axis 5.

For many applications it is sufficient for the first angular speed ω1—with the exception of the acceleration phase at the start of the delay phase at the end—to be constant. In the present case however, in which a heart examination is to be undertaken, that is an examination of a moving organ, this is not the preferred procedure. Instead in this case the first angular speed ω1 is modulated by the control device 6 on the basis of a trigger signal P. The trigger signal P can in this case for example be the recorded pulse beat of the object 10.

As can be seen from the formula in step S2, the first angular speed ω1 is modulated over time t for example according to the formula
ω1(t)=ω0(1+Mcos(π(2t−t1−t2)/T))/2
ω0 is a mean angular speed. M is a modulation factor lying between zero and one, especially between 0.7 and 0.9. It can optionally be determined automatically by control device 6 or specified in advance to the control device 6 by an operator 11.

T is time constant which is determined on the basis of the trigger signal P derived from the object 10. In particular it can be the time between two heart beats. t1 and t2 are phase relationships which will be discussed in greater detail below. The phase relationships t1 and t2 can in this case alternately have the same value or different values.

It can be seen from the above formula that the angular speeds ω1, ω2 are modulated by the control device 6 such that they are maximized in the area of the average value of the phase relationships t1, t2. The modulation in this case, because of the time constant T derived from the trigger signal P, is not only a function of the time t, but is also a function of the trigger signal P.

In a step S3 the control device 6 then checks whether it has received a trigger signal P from the object 10. If it has received such a trigger signal P, in a step S4 it records a first image with the x-ray detector 2 in accordance with the first phase relationship t1 after the trigger signal P at an angular position αi. Likewise, in accordance with the phase relationship t2 after the trigger signal P it records a second image with the second x-ray detector 4 at a second angular position βj.

After the recording of the first and of the second images the control device 6 changes in a step S5 first and second images recorded by x-ray detector-specific corrections. Only then does the control device 6 store the corrected images in a step S6 in a data memory device 12 of the x-ray system.

Next the control device 6 checks, in a step S7, whether the current first angle α is still smaller than the first end angle αE. If it is, the control device 6 continues the operating method with step S2. If not, the control device 6 jumps to a step S8.

As a result of the operating method described above, the control device 6 controls the x-ray unit during the pivoting of the x-ray source 1, 3 and of the x-ray detector 2, 4 such that with first angular positions αi the first x-ray detector 2, records a first image of the object 10 and conveys it to the data memory device 12. Likewise at second angular positions βj a second image of the object 10 is recorded by the second x-ray detector 4 in each case and conveyed to the data storage device 12. The angular positions αi, βj in this case are determined by the control device 6 on the basis of the trigger signals P and the phase relationships t1, t2.

As a result of the situation in which the first angular speed ω1 and thereby also the second angular speed ω2 are modulated and the maximum of the angular speeds ω1, ω2 lies in the middle between the phase relationships t1, t2, the x-ray units during the average of the phase relationships t1, t2 after the trigger signal P cover a relatively large angle. If images are thus not only recorded exactly at the times determined by the phase relationships t1, t2 but at intervals around the phase relationships t1, t2, by means of the modulation of the angular speeds ω1, ω2 described above, the angular area in particular can also be maximized within which the first and second images are recorded.

In step S8 the control device 6 first ends the pivoting of the two x-ray units. Then it checks, in a step S9 whether one of the first angular positions αi matches one of the second angular positions βj. If it does, the control device 6 compares in a step S10 the images recorded at these matching angular positions αi, βj. If the images deviate from each other by more than one image deviation limit δ1, it issues a warning in a step S11. The warning can for example be output to the operator 11, so that it can be perceived immediately by the operator 11 with his sensory organs. Alternately or additionally however, it is also possible for only a warning signal that can be evaluated by a data processing system to be output. For example a corresponding message can be stored together with the first and second images in the data storage device 12. The steps S9 to S11 are of course only executed in this case if the phase relationships t1, t2 are the same or if phase relationships t1, t2 are not involved.

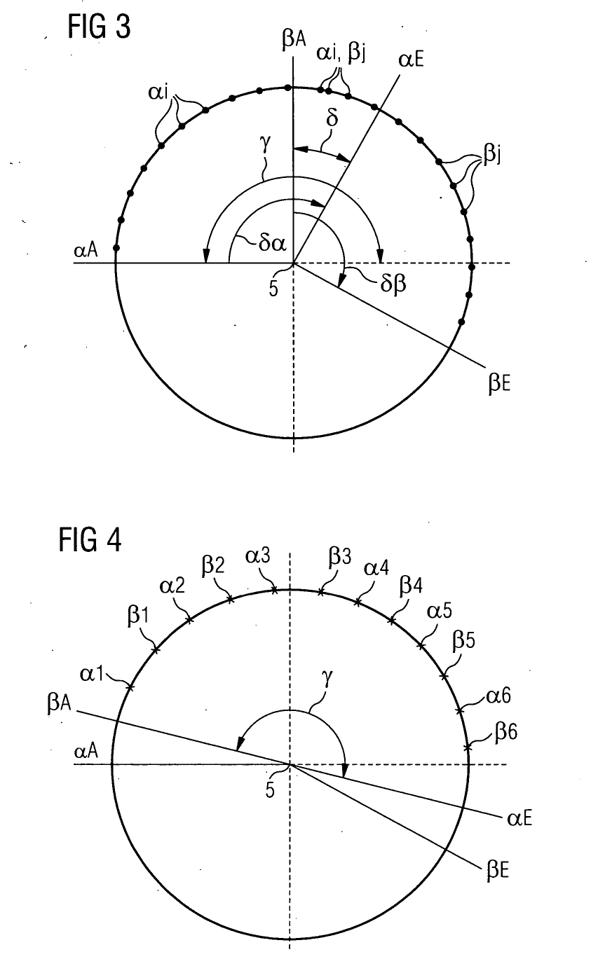

As a rule, 3D reconstruction of the object 10 on the basis of the recorded first and second images in accordance with FIG. 3 requires that images are available from an angular area, which is greater than a limit angle γ. For use of what is known as the Feldkamp algorithm for example the limit angle amounts to γ 180°. Since in the present case however more than just one x-ray unit is available, namely two x-ray units, it is however not absolutely necessary for each of these x-ray units to be able to be pivoted by the limit angle γ. Instead—see for example FIG. 3—a 3D reconstruction of the object 10 is also possible when both the first pivot angle δα and also the second pivot angle δβ are smaller than the limit angle γ, from which a 3D reconstruction of the object 10 is possible. For this however, in accordance with FIG. 3 it is necessary that the pivot angles δα, δβ do not overlap at least partly and that the sum of the pivot angles δα, δβ, if necessary minus an overlapping angle δ, is at least as large as the limit angle γ. In accordance with FIG. 3 the pivot angles δα, δβ can each be 120° for example and overlap by an angle δ of 30°. In this case it is possible to determine a joint 3D reconstruction of the object 10 in which both the first and also the second images are included.

Of course it is however also possible—see FIG. 4 to 6—for the first pivot angle δα and/or the second pivot angle δβ to be at least as large as the limit angle γ. In this case a 3D reconstruction of the object 10 can also be determined on the basis of the first images viewed in themselves or the second images viewed in themselves.

The angular areas covered by the x-ray units are preferably not identical. The start angle αA, βA and/or the end angle αE, βE are also preferably not the same. This applies regardless of whether the size of the angular areas covered, that is the amounts of the pivot angle δα, δβ, are identical. It is also preferably such that the first pivot angle δα and the second pivot angle δβ at least partly do not overlap.

In accordance with FIG. 4 it is possible for example for the pivot angle δα, δβ to be selected so that, between each two immediately consecutive first angular positions αi one of the second angular positions βj is arranged and conversely between each two immediately consecutive second angular positions βj one of the first angular positions αi is arranged in each case. Difference angles of the first angular positions αi to the immediately adjacent second angular positions βj are approximately the same in this case. Likewise the difference angle of the second angular positions βj to the immediately adjacent first angular positions is about the same. In particular the difference angles should thus amount to between 40 and 60% of the angle between each two directly adjacent first or second angular positions αi, βj.

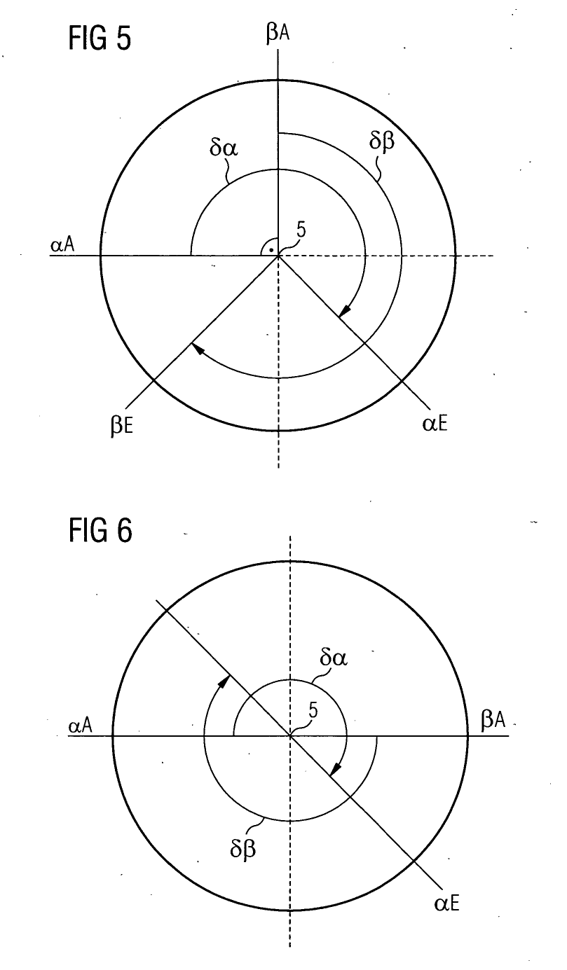

In accordance with FIG. 5 the pivot angles δα, δβ are each 225°. According to FIG. 5 they are offset from each other by an angle of 90°. With this arrangement the first pivot angle δα and the second pivot angle δβ together already cover 315° and thus almost a full circle around the pivot axis 5. With a suitable arrangement and embodiment of the x-ray unit, as shown in FIG. 6, it is even possible for the pivot angles δα, δβ together to cover a complete full circle around the pivot axis 5.

Regardless of the concrete embodiment of the x-ray unit and its pivot angles δα, δβ, the pivot angles δα, δβ and the angular positions αi, βj are however always determined such on the basis of the first and second images of the object 10 recorded by the x-ray detectors 2, 4 always at least one 3D reconstruction of the object 10 can be determined.

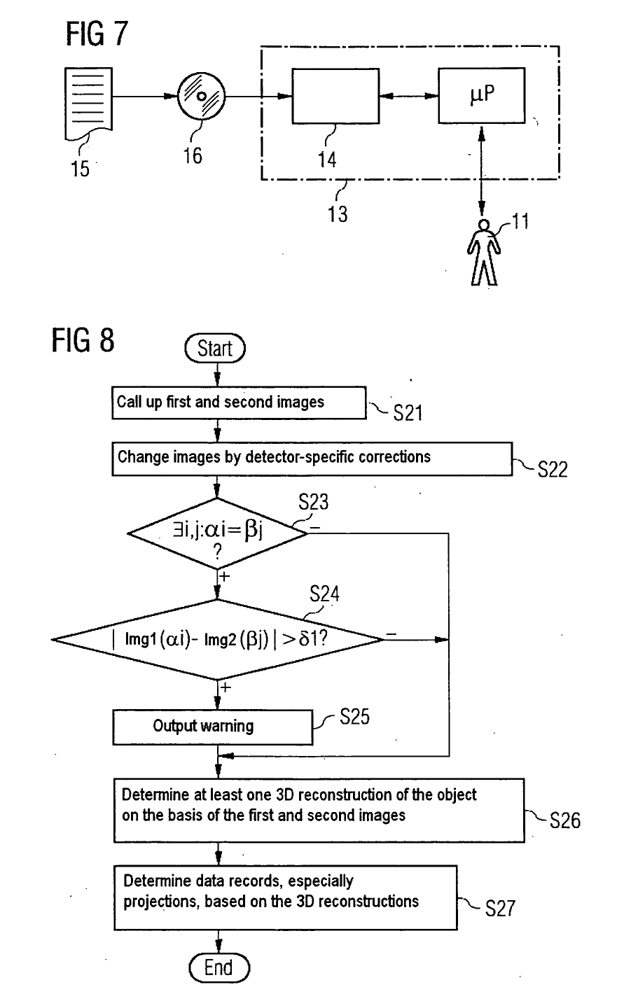

The recorded images are actually evaluated by means of a computer 13, which is shown in greater detail in FIG. 7. The computer 13 can in this case be identical to the control device 6 of the x-ray system of FIG. 1. But it can also be a different device from the control device 6.

The computer 13 includes a mass storage device 14 in which the determination program 15 is stored. The determination program 15 in this case has been conveyed to the computer 13 beforehand via a data medium 16 is which the determination program 15 has also been stored. A typical example of such a data medium 16 is a CD-ROM 16. The determination program 15 could however also have been conveyed to the computer 13 in another way, e.g. via a computer network.

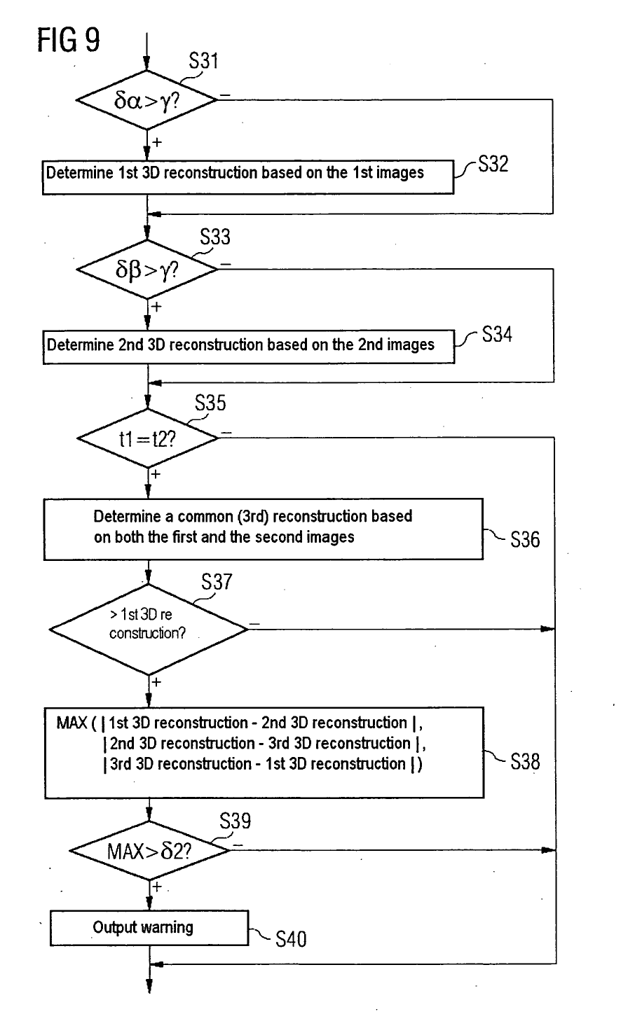

When the determination program 15 is called, the computer 13 executes a transfer procedure for at least one 3D reconstruction of the object 10 on the basis of the first and second images of the object 10, which have been recorded on the basis of the operating method described above for the x-ray system. This determination procedure is described below in greater detail in connection with FIG. 8.

In accordance with FIG. 8 the computer 13, in a step S21, initially retrieves the first and second images from the mass storage device 14. Should the control device 6 not have further corrected the first and second images by x-ray detector-specific corrections, the computer 13 makes these corrections itself in a step S22. Step S22 is thus designed in entirely the same way as step S5 of FIG. 2.

Then the computer 13, in a step S23 checks whether at least one of the first angular positions αi matches one of the second angular positions βj. If it does, the computer 13 compares, in a step S24 the images recorded at these matching angular positions αi, βj. If the two images deviate from each other by more than the image deviation limit δ1, the computer 13 issues a warning in a step S25. The design of steps S23 to S25 is thus entirely the same as that of steps S9 to S11 of FIG. 2.

Then the computer 13 in a step S26 determines on the basis of both the first and also the second images of the object 10 at least one 3D reconstruction of the object 10. This step S26 will be explained in greater detail later in connection with FIG. 9.

After determining the at least one 3D reconstruction the computer 13 determines, depending on the corresponding inputs of a user 17 (preferably two-dimensional) data records of the 3D reconstructions. The two-dimensional data records can in particular be sections, parallel projections or perspective projections. Other types of evaluations, for example histograms, are possible however.

The design of step S26 of FIG. 8 will now be explained in greater detail in connection with FIG. 9.

In accordance with FIG. 9 the computer 13, in a step S31, first checks whether the first pivot angle δα is greater than the limit angle γ. If it is, the computer 13 in a step S32, determines exclusively on the basis of the first images a first 3D reconstruction of the object 10.

Similarly the computer 13 then checks in a step S33 whether the second pivot angle δβ is greater than limit angle γ If it is, the computer 13, in a step S34, determines exclusively on the basis of the second images a second 3D reconstruction of the object 10.

Then computer 13 checks, in a step S35 whether the phase relationships t1, t2 are the same. If they are, the computer 13 determines in a step S36 both on the basis of the first and also of the second images a common 3D reconstruction of the object 10. In particular this 3D reconstruction is—for the same phase relationship t1, t2—always possible, since at least the sum of the two pivot angles δα, δβ, if necessary minus the overlapping angle δ, is at least as large as the limit angle γ. But of course this 3D reconstruction is also possible, if the first and/or the second pivot angle δα, δβ is at least as large as the limit angle γ. This applies especially regardless of whether and to what extent the two pivot angles δα, δβ overlap—see FIG. 3 to 6.

After executing step S36, the computer 13, in a step S37, checks whether, in addition to step S36, it has also executed at least one of the steps S32 and S34, i.e. it has determined more than one 3D reconstruction of the object 10. If it has, the computer 13, in a step S38, determines the difference between the 3D reconstructions that it has determined and from these differences in their maximum. In a step S39, the computer 13 checks whether the maximum value of these differences exceeds a reconstruction deviation limit 62. If it does, the computer 13—, as in steps S11 and S25 of FIGS. 2 and 8—issues a warning in a step S40. The warning can be output here either back to the user 17 but also within the computer.

The above-mentioned comparison determined between a number of 3D reconstructions is only worthwhile if the phase relationships t1, t2 are either the same or (under exceptional circumstances) the phase relationships t1, t2 can be ignored. Then, especially with a moving organ such as the heart of the person 10 the two 3D reconstructions created on the basis of only the first or only the second images of course do not match. However, for this, a 3D reconstruction of the heart in two different states is possible by means of the determination procedure in accordance with the invention.

1-32. (canceled)

33. A method of operating an x-ray system having a control device, a data storage device, a first and a second x-ray unit, the first x-ray unit including a first x-ray source and a first x-ray detector, the first x-ray source and the first x-ray detector configured to be pivoted around a pivot axis and arranged opposite to each other in relative to the pivot axis, the second x-ray unit including a second x-ray source and a second x-ray detector, the second x-ray source and the second x-ray detector configured to be pivoted around the pivot axis and arranged opposite to each other relative to the pivot axis, wherein an examination object is arranged at or adjacent to the pivot axis, the method comprising:
pivoting the first x-ray source and the first x-ray detector around the pivot axis by a first pivot angle and simultaneously pivoting the second x-ray source and the second x-ray detector around the pivot axis by a second pivot angle using the control device;
capturing a plurality of first images of the examination object at a plurality of first angular positions by the first x-ray detector while pivoting the first and second x-ray sources and x-ray detectors;
storing the first images in the data storage device;
capturing a plurality of second images of the examination object at a plurality of second angular positions by the second x-ray detector while pivoting the first and second x-ray sources and x-ray detectors;
storing the second images in the data storage device; and
reconstructing a three-dimensional image of the examination object based on the first and second images.

34. The method in accordance with claim 33, wherein
at least one of the first angular positions matches at least one of the second angular positions,
the control device compares such first images captured at the matching angular position with such second images captured at the matching angular position, and
the control device outputs a warning if the compared images differ.

35. The method in accordance with claim 33, wherein
the first and second pivot angles are each smaller than a limit angle, the limit angle defined as the minimum angle allowing for reconstructing the three-dimensional image,
the first and second pivot angle overlap at most by an overlapping angle, and
an angle sum calculated by adding the first and second pivot angles and subtracting the overlapping angle at least equals the limit angle.

36. The method in accordance with claim 33, wherein the first or the second pivot angle is at least as large as a limit angle, the limit angle defined as the minimum angle allowing for reconstructing the three-dimensional image.

37. The method in accordance with claim 33, wherein the first and second pivot angles cover a full circle around the pivot axis.

38. The method in accordance with claim 33, wherein
the first and second x-ray detectors and the first and second x-ray sources are pivoted around the pivot axis by the control device with a first respectively a second angular speed, the second angular speed based on the first angular speed.

39. The method in accordance with claim 33, wherein the first and second angular positions are determined by the control device upon a trigger signal derived from the examination object.

40. The method in accordance with claim 39, wherein a first and second angular speeds with which the first respectively second x-ray units are pivoted around the pivot axis is modulated by the control device based on the trigger signal.

41. An x-ray system, comprising:
a control device;
a data storage device;
a first and a second x-ray unit, the first x-ray unit having a first x-ray source and a first x-ray detector, the first x-ray source and the first x-ray detector configured to be pivoted around a pivot axis and arranged opposite to each other relative to the pivot axis, the second x-ray unit having a second x-ray source and a second x-ray detector, the second x-ray source and the second x-ray detector configured to be pivoted around the pivot axis and arranged opposite to each other relative to the pivot axis; and
an examination object arranged at or adjacent to the pivot axis, wherein the control device is configured to:
pivot the first x-ray source and the first x-ray detector around the pivot axis by a first pivot angle and simultaneously pivot the second x-ray source and the second x-ray detector around the pivot axis by a second pivot angle;
trigger capturing a plurality of first images of the examination object at a plurality of first angular positions by the first x-ray detector while pivoting the first and second x-ray sources and x-ray detectors;
trigger storing the first images in the data storage device;
trigger capturing a plurality of second images of the examination object at a plurality of second angular positions by the second x-ray detector while pivoting the first and second x-ray sources and x-ray detectors;
trigger storing the second images in the data storage device; and
reconstruct a three-dimensional image of the examination object based on the first and second images.

42. The x-ray system in accordance with claim 41, wherein
at least one of the first angular positions matches at least one of the second angular positions,
the control device compares such first images captured at the matching angular position with such second images captured at the matching angular position, and
the control device outputs a warning if the compared images differ.

43. The x-ray system in accordance with claim 41, wherein
the first and second pivot angles are each smaller than a limit angle, the limit angle defined as the minimum angle allowing for reconstructing the three-dimensional image,
the first and second pivot angle overlap at most by an overlapping angle, and
an angle sum calculated by adding the first and second pivot angles and subtracting the overlapping angle at least equals the limit angle.

44. The x-ray system in accordance with claim 41, wherein the first or the second pivot angle is at least as large as a limit angle, the limit angle defined as the minimum angle allowing for reconstructing the three-dimensional image.

45. The x-ray system in accordance with claim 41, wherein the first and second pivot angles cover a full circle around the pivot axis.
